(19) United States
(12) Patent Application Publication
(10) Pub. No.: US 2022/0008514 A1
(43) Pub. Date: Jan. 13, 2022

(54) COMBINED USE OF SELECTIVE SEROTONIN REUPTAKE INHIBITORS AND HEMATOPOIETIC GROWTH FACTORS FOR TREATING HEMATOPOIETIC DISEASES

(71) Applicants: IMAGINE INSTITUT DES MALADIES GENETIQUES NECKER ENFANTS MALADES, Paris (FR); ASSISTANCE PUBLIQUE-HOPITAUX DE PARIS, Paris (FR); CENTRE NATIONAL DE LA RECHERCHE SCIENTIFIQUE, Paris (FR); INSTITUT GUSTAVE ROUSSY, Villejuit (FR); INSTITUT NATIONAL DE LA SANTE ET DE LA RECHERCHE MÉDICALE, Paris (FR); UNIVERSITE DE PARIS, Paris (FR)

(72) Inventors: Tereza COMAN, Paris (FR); Francine COTE, Paris (FR); Olivier HERMINE, Paris (FR); Guillemette FOUQUET, Paris (FR); Julien ROSSIGNOL, Paris (FR)

(21) Appl. No.: 17/297,546

(22) PCT Filed: Nov. 28, 2019
(86) PCT No.: PCT/EP2019/083002

§ 371 Date: May 27, 2021

(30) Foreign Application Priority Data
Nov. 28, 2018 (EP) 18306579.6

(51) Int. Cl. A61K 38/20; A61K 31/138; A61K 31/343; A61K 31/135; A61K 31/4525; A61K 31/15; A61K 38/18; A61K 38/19; A61P 7/06

(52) U.S. Cl. CPC A61K 38/204; A61K 31/138; A61K 31/343; A61K 31/135; A61K 31/4525; A61P 7/06; A61K 38/1816; A61K 38/196; A61K 38/193; A61K 38/202; A61K 38/1858; A61K 31/15

(57) ABSTRACT

The invention relates to the combined use of selective serotonin reuptake inhibitors (SSRIs) and hematopoietic growth factors as a drug and particularly for treating cytopenia related to hematopoietic diseases or chemotherapy, and also to a pharmaceutical kit comprising both SSRIs and hematopoietic growth factors. This combination is more particularly used for treating patients presenting cytopenia, and patients in need of chemotherapy and more particularly to reduce length of chemotherapy-induced aplasia.

### FILED OF THE INVENTION

The present invention relates to the field of hematopoietic diseases involving cytopenia related to hematopoietic stem and progenitor cells disorders, or chemotherapy with or without hematopoietic stem cells transplantation. It relates to the combined use of selective serotonin reuptake inhibitors (SSRIs) and hematopoietic growth factors as a drug and particularly for treating these diseases, and also to a pharmaceutical kit comprising both SSRIs and hematopoietic growth factors. The combination is more particularly used for treating patients presenting cytopenia due to hematopoietic diseases and patients in need of chemotherapy and more particularly to reduce length of aplasia after chemotherapy or radiotherapy.

### BACKGROUND OF THE INVENTION

Hematopoietic stem cells (HSCs) are the stem cells responsible for the production of mature blood cells in bone marrow. This process is called hematopoiesis and the only way by which all mature blood cells are produced. It must balance enormous production needs (more than 500 billion blood cells are produced every day) with the need to precisely regulate the number of each blood cell type in the circulation. The vast majority of hematopoiesis occurs in the bone marrow and is derived from a limited number of hematopoietic stem cells (HSCs) that are multipotent and capable of extensive self-renewal.

HSCs give rise to both the myeloid and lymphoid lineages of blood cells. Myeloid cells include monocytes to macrophages, neutrophils, basophils, eosinophils, erythrocytes, and megakaryocytes to platelets. Lymphoid cells include T cells, B cells, and natural killer cells. Myeloid and lymphoid lineages are both involved in dendritic cell formation. The hematopoietic tissue contains cells with long-term and short-term regeneration capacities and committed multipotent, oligopotent, and unipotent progenitors. HSCs constitute 1:10.000 of cells in myeloid tissue. HSC transplants are used for example in the treatment of cancers, preferably hematologic cancers and other immune system disorders.

It is well-known in the art, especially in hematology, that stem and progenitor cells are widely used for treatments such as bone marrow or peripheral stem cell transplantation. Majority of autograft and 75% of allograft of hematopoietic stem cells (HSCs) are realized with HSCs from peripheral blood stem cells after mobilization.

However, 30% of donors are in failed state after mobilization (L. B. et al. 1992; L. B., Dyson, P. G. & Juttner, C. 1986). One strategy envisaged for treating these pathologies is to improve mobilization of peripheral blood stem cells from donors. Consequently, in the context of hematopoietic diseases, there is a need to identify new factors improving mobilization of peripheral blood stem cells from donors and thus, a better management of patients suffering from hematopoietic diseases, particularly hemopathy. This refers to needs of improving the output of chemotherapy, especially chemotherapy-induced aplasia by reducing the duration of aplasia.

Previous studies mentioned that serotonin (5-hydroxytryptamine or 5-HT) plays a role in embryogenic development, regenerative properties, a role on stem and progenitor cells during development and after, and more specifically, its role in hematopoiesis (Reviewed in Fouquet et al. 2018).

A large number of studies have focused on the role of serotonin as a neurotransmitter in the central nervous system, although only a small percentage of the body's serotonin (˜5%) can be found in the mature brain of mammals (V. Erspamer, 1937; V. Erspamer, B. Asero, 1952). In the gut, the enterochromaffin cells are scattered in the enteric epithelium from the stomach through the colon and produce over 95% of the body's serotonin. Since the generation of tryptophan hydroxylase (Tph1 and Tph2) knockout mice, unsuspected roles have been identified for serotonin which is synthesized outside of the brain (Reviewed in S. N. Spohn, G. M. Mawe, 2017; P. Amireault et al. 2013; Mosienko V et al. 2015).

The murine model deficient in peripheral serotonin (Tph^{-/-}) is a unique experimental tool for exploring the molecular and cellular mechanisms involving serotonin's local effects through microserotonergic systems. The inventors previously showed and described the role of peripheral serotonin on stem cells as well as on hematopoietic progenitors, especially the role of serotonin in hematopoietic diseases, and whether targeting the serotonergic system could be of therapeutic value for the regulation of normal and pathological hematopoiesis (Fouquet et al. 2018). Further, it is known in the art that treatments with selective serotonin reuptake inhibitors (SSRIs) such as fluoxetine, by blocking the action of SERT, lead to increased 5-HT extracellular concentrations and signaling (J. P. Feighner, 1999).

Furthermore, if each of the molecules has been tested alone in many diseases involving or not hematopoietic diseases, no research team has up to now suggested combining selective serotonin reuptake inhibitors (SSRIs) and hematopoietic growth factors in the context of the treatment of any of these diseases.

### SUMMARY OF THE INVENTION

In the context of the invention, the inventors surprisingly found that the combined use of selective serotonin reuptake inhibitors (SSRIs) and hematopoietic growth factors generates surprising or synergistic effects, thus making it possible to improve the treatment of patients suffering from cytopenia related to hematopoietic diseases or chemotherapy and can be used as a drug, preferably to improve cytopenia and after a chemotherapy or radiotherapy to reduce the length of aplasia.

In a first aspect, the present invention thus relates to a combination of at least one selective serotonin reuptake inhibitor (SSRI) and at least one hematopoietic growth factor for use as drug.

Indeed, inventors provides experimental data showing that said combinations have beneficial and unexpected effects toward hematopoietic stem and progenitor cells. The invention thus further relates to a combination of at least one selective serotonin reuptake inhibitor (SSRI) and at least one hematopoietic growth factor for use to improve hematopoietic stem and progenitor cell regeneration in a subject in need thereof.

In a preferred embodiment, said subject is suffering from cytopenia. Cytopenia can occur after several hematopoietic diseases or chemotherapy. Accordingly, treatment of said diseases can involve a hematopoietic stem cell transplantation. Consequently, invention also relates to the combination of at least one selective serotonin reuptake inhibitor (SSRI) and at least one hematopoietic growth factor is for use to improve hematopoietic stem and progenitor cell regeneration in a subject in need for hematopoietic stem cell transplantation, more specifically in a subject who has been subjected to high doses and/or myeloablative chemotherapy and/or TBI in order to eliminate disease or cancer and/or ensure stem cell engraftment. Accordingly, combinations of the invention are of use in allograft (e.g. hematopoietic stem cells (HSCs)-allograft with cells originating from bone marrow, cord blood or peripheral blood from a donor) or autograft context (e.g. a peripheral blood hematopoietic stem cells (HSCs)-autograft). Also, data from the experimental data show that, in that context, combinations of the invention are of particular use in improving hematopoietic stem and progenitor cells mobilization (donor or recipient) and/or engraftment function in a subject (recipient), which constitutes a particular embodiment of the invention.

Hence, combination of the invention is particularly advantageous for improving mobilization of Hematopoietic Stem Cells in donor, from which they are collected in order to be transplanted in graft receiver.

Besides HSCs transplantation, invention also relates to a combination at least one selective serotonin reuptake inhibitor (SSRI) and at least one hematopoietic growth factor for use in treating cytopenia secondary to chimiotherapy or radiotherapy or secondary to a hematopoietic disease.

In a specific embodiment, said hematopoietic disease can be a malignant hemopathy, for example, myelodysplastic syndromes (MDS), aplastic anemia, myeloproliferative neoplasm, acute leukemia. In another embodiment, said hematopoietic disease can be a non-malignant hemopathy, for example hemolytic anemia, hemoglobinopathies, inherited or acquired peripheral thrombopenia, inherited or acquired neutropenia.

The invention further relates to a combination of selective serotonin reuptake inhibitors (SSRIs) and hematopoietic growth factors for use in the treatment of hematopoietic diseases.

The unexpected and synergistic improvement of hematopoietic stem and progenitor cell regeneration resulting from the administration of combination of at least one selective serotonin reuptake inhibitor (SSRI) and at least one hematopoietic growth factor is particularly advantageous and allows to reduce length of aplasia, transfusion needs, aplasia related infections, and, more generally, improve quality of life of subject suffering of said diseases or condition.

In a preferred embodiment, selective serotonin reuptake inhibitors (SSRIs) is selected from the group consisting in fluoxetine, citalopram, sertraline, paroxetine, escitalopram, fluvoxamine and hematopoietic growth factors is selected from the group consisting in erythropoietin (EPO), thrombopoietin (TPO), granulocyte colony-stimulating factor (G-CSF), granulocyte-macrophage colony-stimulating factor (GM-CSF), macrophage colony-stimulating factor (M-CSF), platelet-derived growth factor (PDGF), interleukin 3 (IL-3), interleukin 6 (Il-6).

In another aspect, the invention also relates to a pharmaceutical kit comprising selective serotonin reuptake inhibitors (SSRIs) and hematopoietic growth factors, preferably for use as drug. Selective serotonin reuptake inhibitors (SSRIs) and hematopoietic growth factors can be present in the kit in a single formulation or in two distinct formulations.

The combination of the invention is particularly suited to improve Hematopoietic Stem Cells mobilization and/or engraftment function, to improve cytopenia and reduce the length of aplasia, preferably after chemotherapy or radiotherapy. Therefore, the invention is particularly suited for treating diseases associated to hematopoietic disorders and/or for treating cytopenia.

### LEGEND OF DRAWING

FIG. 1. Cell-autonomous action of serotonin contributes in vivo to normal erythropoiesis (mouse). Identification of components of the 5-HT system in murine progenitor cells of the bone marrow and presence of mRNA for Tph1 selectively and highly expressed at the CFU-E-to-pro-erythroblast transition checkpoint (CD71⁺/c-Kit⁺/TER119⁻ to CD71⁺/c-Kit⁻/TER119⁻) (FIG. 1A). High levels of mRNA expression encoding for SERT (FIG. 1B) and 5-HT_{2A}R (FIG. 1C) were also seen specifically in the pro-erythroblast population (CD71⁺/c-Kit⁻/TER119⁻) population, derived from the bone marrow of adult WT mice (2-month-old). 5-HT plays a cell-autonomous role as the anemic phenotype was transplantable by Tph1^{-/-} bone marrow cells in WT mice. Specifically, WT mice that received a transplant from Tph1^{-/-} (KO/WT) bone marrow cells had decreased Hb levels, whereas Tph1^{-/-} mice that received a transplant from WT bone marrow cells (WT/KO) presented no anemic phenotype (D). Tph1 is positively regulated by EPO in a dose-dependent manner (FIG. 1E) and 5-HT synthesis occurred as early as 3 hours following EPO stimulation (FIG. 1F). Increased EPO concentration also up-regulated 5-HT_{2A}R (FIG. 1G) but downregulated SERT (FIG. 1H). EPO stimulates 5-HT synthesis in pro-erythroblasts, up-regulates the 5-HT_{2A}R and down regulates SERT to increase extra-cellular concentrations of 5-HT and prolonged its action via the 5-HT_{2A}R. (data are presented as mean±SEM. Paired and an unpaired t-tests were used when appropriate. **P<0.005, ***P<0.0005)

FIG. 2. Complete serotonergic system in human erythroid progenitors. In purified human cord blood cells, using RT-qPCR, inventors demonstrate that TPH1(FIG. 2A), the 5-HT2A receptor (5-HT_{2A}R-HTR2a, FIG. 2B) and the 5-HT specific membrane transporter (SERT-slc6a4, FIG. 2C) were highly expressed: In human CD36⁺ cord blood cells cultured with EPO; TPH1, HTR2a, and SLC6a4 genes are found expressed at the pro-erythroblast stage of differentiation (from day 3 of culture after CD36⁺ isolation (Zermati et al., 2001). (data are presented as mean±SEM. Paired and an unpaired t-tests were used when appropriate. ***P<0.0005)

FIG. 3. Human in vitro experiments (effect of 5-HT combined with EPO). To determine the role played by serotonin in erythroid proliferation, inventors used a well-defined culture system that closely mimics the proliferation and differentiation of erythroid precursors in vivo. The use of 5-HT (FIG. 3A) or PNU 22394 (FIG. 3B), a 5-HT_{2A}R agonist, significantly enhances erythroid proliferation. Erythroid cells were generated from CD34⁺ cord blood progenitor cells in serum-free medium in the presence of EPO (2 mU/ml)+IL-3 (10 ng/ml)+stem cell factor (SCF; 50 ng/ml). Throughout data are mean±SEM. Unpaired t-test and Pearson linear and non-linear regressions were used when appropriate. *P<0.05, **P<0.005, ***P<0.0005.

FIG. 4. Mice in vivo experiments (effect of 5-HT). Graphical representation of the experimental procedures for experiments 3A, 3B and 3C set up to understand the in vivo mechanism of action of 5-HT signaling on bone marrow cells, in a model of anemia. Prior to sublethal irradiation, WT or Tph^{+/-} mice were administered the well-known SSRI fluoxetine or a placebo.

FIG. 5. Mice in vivo experiments (effect of SSRI). Reticulocytes (%) (FIG. 5A) and Hb levels (g/dl) (FIG. 5B) in WT mice (n=7) treated with placebo or fluoxetine for 7 days followed by sub-lethal irradiation. (Data are from 2 independent experiments). In WT mice treated with placebo, a prolonged anemia without normalization of Hb until day 22 was observed, whereas, in WT mice treated with fluoxetine, a rapid and significant increase in the number of reticulocytes was observed as early as day 3 and a normalization of Hb levels were seen as early as day 11. In Tph1^{+/-} mice where 5-HT levels are 50% of the WT control levels, starting on day 7 following sub-lethal irradiation, it is observed a significant increase in the CD71+/TER119+ population in mice treated with fluoxetine as compared to the ones treated with placebo (FIG. 5C and FIG. 5D). Measurement of 5-HT levels in bone marrow revealed an increase on day 7 of treatment which correlates with the initiation of proliferation (not shown). Hence, restoration of 5-HT levels through SSRI treatment prolongs and increases 5-HT_{2A}R stimulation, which in turn enhances cellular division of erythroid progenitors to rescue the proliferation defect. (FIG. 5C and FIG. 5D). Throughout data are mean±SEM. Paired and an unpaired t-tests were used when appropriate. *P<0.05, **P<0.005, ***P<0.0005.

FIG. 6. Human cohort study (effect of SSRI on chemotherapy induced aplasia). A reduced duration of aplasia is observed in patients treated with SSRI at the time of autologous HSCT (x), in comparison with the duration of aplasia observed for patients having not been under SSRI medication (+).

FIG. 7. Mice in vivo experiments (effect of SSRI+G-CSF on irradiation-induced cytopenia). Hemoglobin (FIG. 7A), platelets (FIG. 7B), white blood cells (FIG. 7C) and polynuclear neutrophils (FIG. 7D) levels following sub-lethal irradiation in 8-week-old WT C57BL/6 female mice. Levels were measured in placebo, SSRI (treatment with fluoxetine from 7 days before irradiation), G-CSF (treatment with G-CSF from day 4 after irradiation), SSRI+G-CSF (treatment with both fluoxetine and G-CSF as described) treated mice (n=5). A potent effect on all myeloid lineages is observed in mice treated with combination of a SSRI and a hematopoietic growth factor of the invention. A rapid increase in hemoglobin (FIG. 7A), platelets (FIG. 7B), white blood cells and polynuclear neutrophils (FIG. 7C and FIG. 7D) is observed for mice treated with the combination of fluoxetine and G-CSF, when compared to mice administered with placebo mice and also to mice treated with G-CSF or fluoxetine alone. Unexpectedly, an improved survival of mice treated with the combination of fluoxetine and G-CSF (100% survival) is observed when compared with the placebo group (0% survival), but also compared with G-CSF or fluoxetine as monotherapy (FIG. 7E).

FIG. 8. Mice in vivo experiments (effect of SSRI+G-CSF on irradiation-induced cytopenia). Hemoglobin (FIG. 8A), platelets (FIG. 8B), and polynuclear neutrophils (FIG. 8C) levels following sub-lethal irradiation in 8-week-old WT C57BL/6 female mice. Levels were measured in placebo, SSRI (treatment with fluoxetine from 7 days before irradiation), G-CSF (treatment with G-CSF from day 4 after irradiation), SSRI+G-CSF (treatment with both fluoxetine and G-CSF as described) treated mice (n=10-13, 3 independent experiments). A potent effect on all lineages is observed in mice treated with combination of a SSRI and a hematopoietic growth factor of the invention. An earlier and rapid increase in hemoglobin (FIG. 8A), platelets (FIG. 8B), polynuclear neutrophils (FIG. 8C) is observed for mice treated with the combination of fluoxetine and G-CSF, when compared to mice administered with placebo mice and also to mice treated with G-CSF or fluoxetine alone. In addition, data from FIG. 8A demonstrate that 100% of mice treated with SSRI+G-CSF have hemoglobin values over the critical anemic threshold, 9 days earlier than mice treated with G-CSF or SSRI alone. The same applies for neutrophils and platelets levels. (data are mean±SEM. Paired and an unpaired t-tests were used when appropriate. *P<0.05, **P<0.005)

FIG. 9. Mice in vivo experiments (effect of SSRI+TPO on irradiation-induced cytopenia). Hemoglobin levels following sub-lethal irradiation in 8-week-old WT C57BL/6 female mice. Levels were measured in placebo, SSRI (treatment with fluoxetine from 7 days before irradiation), TPO (treatment with TPO from day 0 after irradiation), SSRI+TPO (treatment with both fluoxetine and TPO as described) treated mice (n=5, one experiment, data are mean±SEM. Paired and an unpaired t-tests were used when appropriate. *P<0.05).

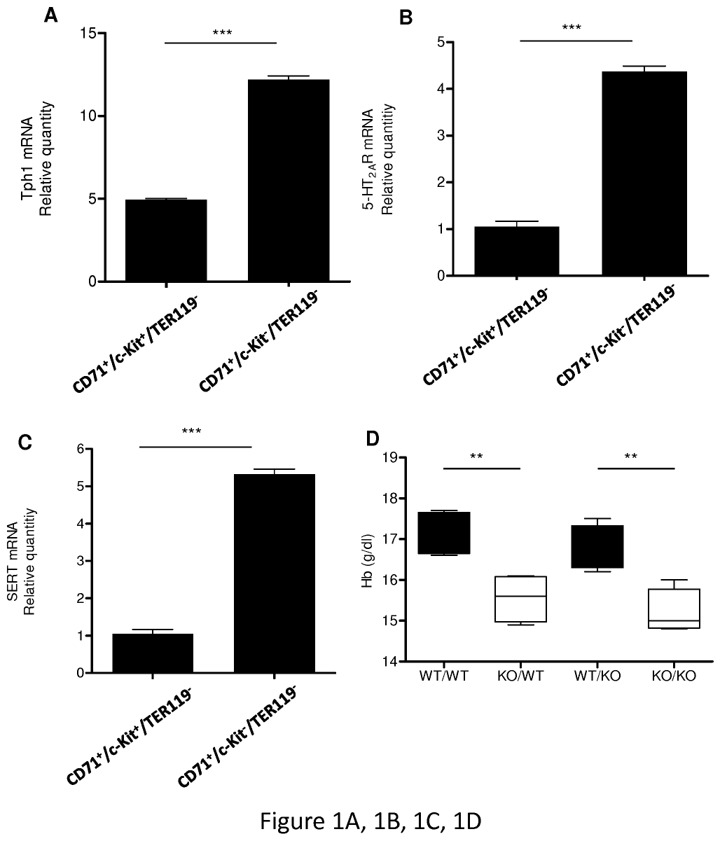

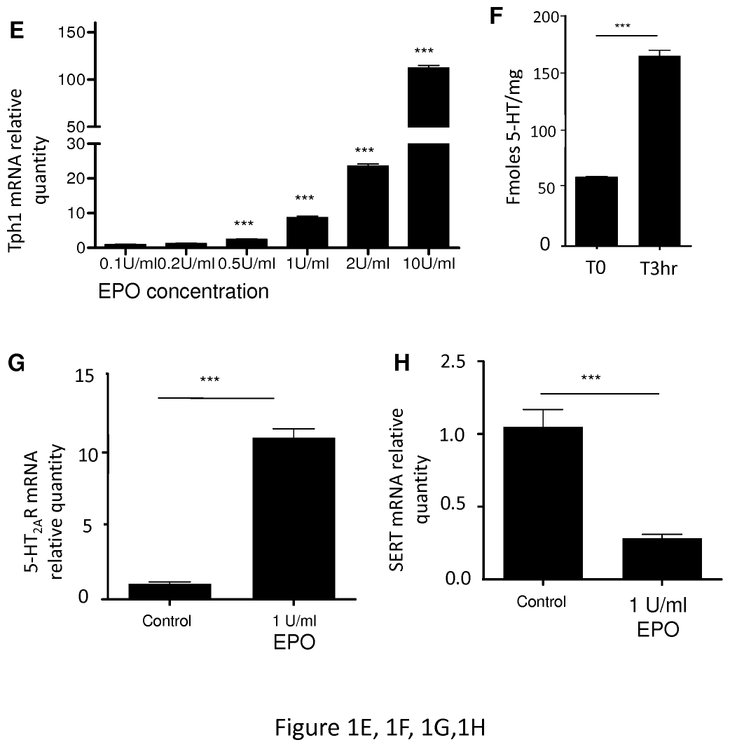

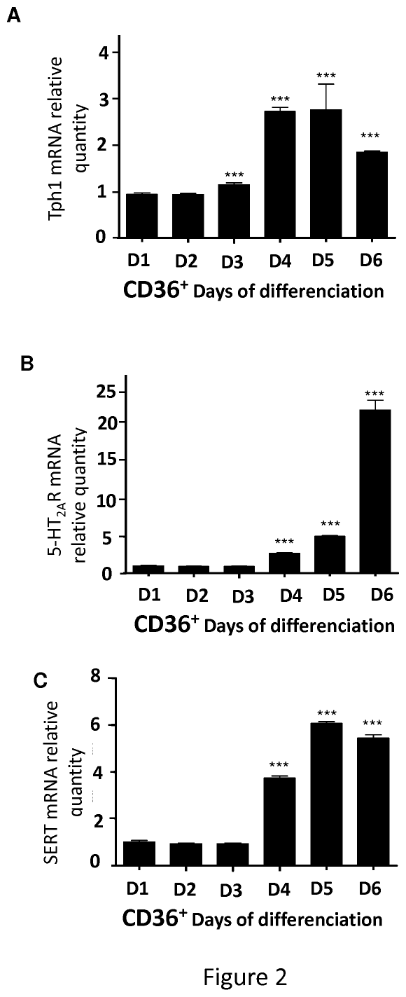

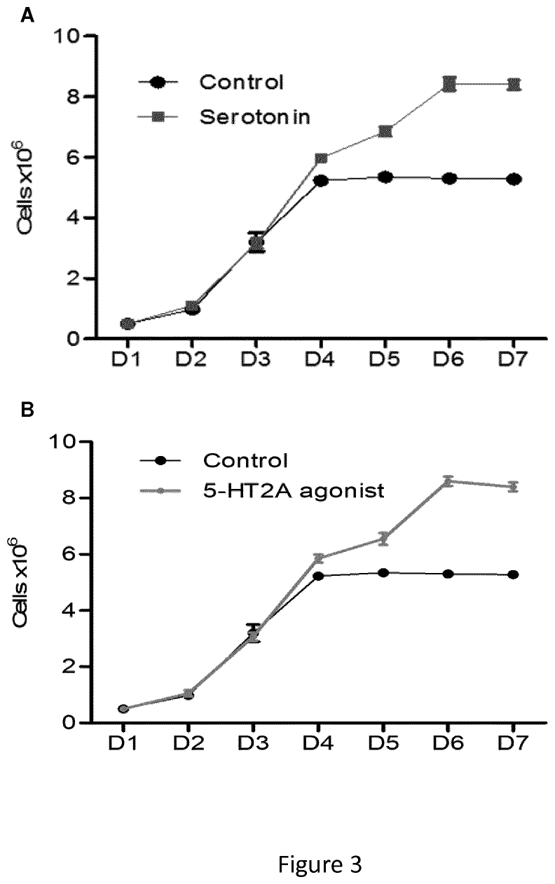

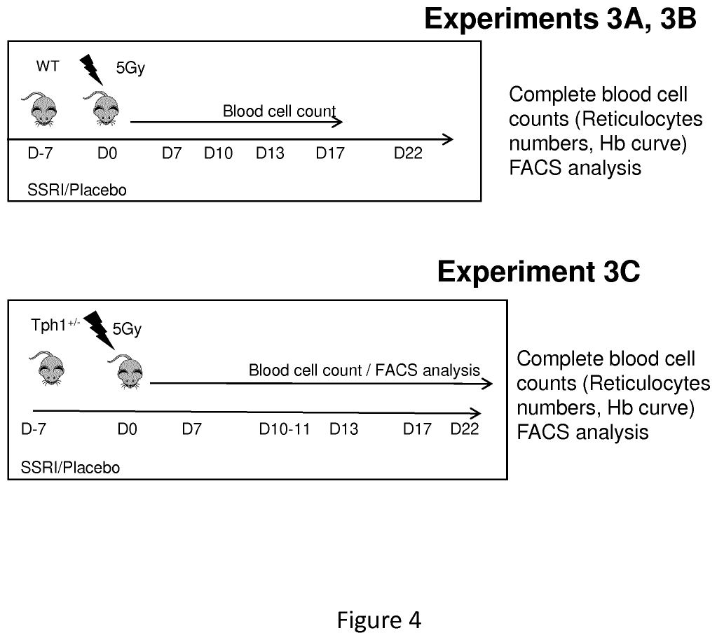

### DETAILED DESCRIPTION OF THE INVENTION

### Definitions

As intended herein, the term “comprising” has the meaning of “including” or “containing”, which means that when an object “comprises” one or several elements, other elements than those mentioned may also be included in the object. In contrast, when an object is said to “consist of” one or several elements, the object cannot include other elements than those mentioned.

According to the invention, the terms “subject”, “host”, “individual”, and “patient” are used interchangeably herein and refer to a mammal affected or likely to be affected with disease associated with hematopoietic diseases and/or cytopenia, being more precisely a mammal presenting cytopenia or in need of chemotherapy, preferably mammal treated with chemotherapy followed by a hematopoietic stem cell transplantation. For example, a mammal suffering from hemopathy. Subjects are preferably a human being, male or female at any age that is in need of a therapy as described herein.

The term “hematopoietic diseases” or hematopoietic disorders refers to diseases that are specifically binds to Hematopoietic System. Preferably, “hematopoiesis” refers to the formation of blood cellular components. All cellular blood components are derived from hematopoietic stem cells (HSCs). In a healthy adult person, approximately 10¹¹-10¹² new blood cells are produced daily in order to maintain steady state levels in the peripheral circulation. For example, and without limiting the scope of the present invention, hematopoietic diseases are hematopoietic diseases responsible for development of anemia of central or peripheral mechanism, hematopoietic diseases responsible for development of thrombopenia of central or peripheral mechanism, hematopoietic diseases responsible for development of neutropenia of central or peripheral mechanism. Preferably, hematopoietic diseases responsible for development of anemia are malignant hemopathies, myelodysplastic syndromes, hemolytic anemia, hemoglobinopathies, or aplastic anemia; hematopoietic diseases responsible for development of thrombopenia are malignant hemopathies, myelodysplastic syndromes, inherited or acquired peripheral thrombopenia, or aplastic anemia; and hematopoietic diseases responsible for development of neutropenia are malignant hemopathies, myelodysplastic syndromes, inherited or acquired neutropenia, or aplastic anemia. Malignant hemopathies, include myelodysplastic syndromes (MDS), aplastic anemia, myeloproliferative neoplasm, acute leukemia. Non-malignant hemopathies include hemolytic anemia, hemoglobinopathies, inherited or acquired peripheral thrombopenia, inherited or acquired neutropenia

Hematopoietic stem cells (HSCs) are in the medulla of the bone (bone marrow) and have the unique ability to give rise to all of the different mature blood cell types and tissues. HSCs are self-renewing cells: when they differentiate, about 50% of their daughter cells remain as HSCs, so stem cells are not depleted. The other 50% of daughters of HSCs (myeloid and lymphoid progenitor cells) can follow any of the other differentiation pathways that lead to the production of one or more specific types of blood cell. All blood cells are divided into two lineages: myeloid and lymphoid.
Cells of the myeloid lineage, which include erythroblasts and red blood cells, also called erythrocytes, megakaryocytes and platelets, granulocytes, macrophages and monocytes, are derived from common myeloid progenitors, and are involved in such diverse roles as oxygen transport, innate immunity and blood clotting. Erythrocytes are functional and are released into the blood. The number of reticulocytes, immature red blood cells, gives an estimate of the rate of erythropoiesis. This is myelopoiesis.
Lymphocytes derived from common lymphoid progenitors. The lymphoid lineage is composed of T-cells, B-cells and natural killer cells. This is lymphopoiesis.

As used herein, the term “Hematopoietic stem cell transplantation” or “HSCT” is the transplantation of hematopoietic stem cells, usually derived from bone marrow, peripheral blood, or umbilical cord blood. It may be autologous (the patient's own stem cells are used), allogeneic (the stem cells come from a non-identical donor of the same species) or syngeneic (from an identical twin). It is most often performed for patients with certain cancers of the blood (hemopathy) or bone marrow, such as multiple myeloma or leukemia. In these cases, the recipient's immune system is usually destroyed with radiation or chemotherapy before the transplantation. Infection and graft-versus-host disease are major complications of allogeneic HSCT.

The term “chemotherapy” refers to a treatment that uses drugs or radiation, preferably to stop the growth of cancer cells, either by killing the cells or by stopping them from dividing. Chemotherapy may be given by mouth, injection, or infusion, or on the skin, depending on the used. It may be given alone or with other treatments, such as surgery, radiation therapy, or biologic therapy. Alternately, treatment is given to patient in need of HSCT.

The term “Graft-Versus-Host Disease (GVHD)” refers to a common and serious complication wherein there is a reaction of donated immunologically competent lymphocytes against a transplant recipient's own tissue. GVHD is a possible complication of any transplant that uses or contains stem cells from either a related or an unrelated donor.

Consequently, Hematopoietic stem cell transplantation remains a dangerous procedure with many possible complications; it is reserved for patients with life-threatening diseases. As survival following the procedure has increased, its use has expanded beyond cancer to autoimmune diseases and hereditary skeletal dysplasias; notably malignant infantile osteopetrosis and mucopolysaccharidosis.

The term “cancer” as used herein is defined as disease characterized by the rapid and uncontrolled growth of aberrant cells. Cancer cells can spread locally or through the bloodstream and lymphatic system to other parts of the body. In the context of the invention cancer is preferably hematopoietic disease and more preferably a hematopoietic malignancy. Hematopoietic diseases as used herein is defined as being responsible for development of anemia of central or peripheral mechanism, being responsible for development of thrombopenia of central or peripheral mechanism, and/or being responsible for development of neutropenia of central or peripheral mechanism. Hematopoietic malignancy refers to tumors that affect the blood, bone marrow, lymph, and lymphatic system including lymphoid organs. All those elements are all intimately connected through both the circulatory system and the immune system, a disease affecting one will often affect the others as well, making myeloproliferation and lymphoproliferation, and thus the leukemias and the lymphomas.

Hematopoietic malignancies derive from either of the two major blood cell lineages: myeloid and lymphoid cell lines. Lymphomas, lymphocytic leukemias, and myeloma are from the lymphoid line, while acute and chronic myelogenous leukemia, myelodysplastic syndromes and myeloproliferative diseases are myeloid in origin.

“Myelodysplastic syndrome” or “MDS” refers to a group of cancers in which immature blood cells in the bone marrow do not mature and therefore do not become healthy blood cells. Symptoms may include feeling tired, shortness of breath, easy bleeding, or frequent infections. Risk factors include previous chemotherapy or radiation therapy, exposure to certain chemicals such as tobacco smoke, pesticides, and benzene, and exposure to heavy metals such as mercury or lead. In about 1 in 3 patients, MDS can progress to a cancer of bone marrow cells called acute myeloid leukemia (AML). Because all patients do not get leukemia, MDS used to be classified as a disease of low malignant potential. But now that we have learned more, MDS is considered to be a form of cancer. Treatments may include supportive care, drug therapy, and stem cell transplantation.

In the case of stem cell transplantation and particularly hematopoietic stem cell transplantation, there is a previous step of mobilization of HSCs. Hematopoietic stem cells (HSCs) normally reside in the bone marrow but can be forced into the blood. “Mobilization of HSCs” refers to this process which are used clinically to harvest large numbers of HSCs for transplantation. Currently, the mobilizing agent of choice is granulocyte colony-stimulating factor. However, not all subjects mobilize well, and combinations of the invention comprising at least one selective serotonin reuptake inhibitor (SSRI) and at least one hematopoietic growth factor provide, as described herein, a valuable tool to improve mobilization of HSCs in donors. Also, mobilization of HSCs is also used when referring to the use of combined treatment of the invention for treating cytopenia secondary to hematopoietic diseases and/or chemotherapy and/or radiotherapy.

In the context of the invention, “Engraftment function” occurs after autologous HSCT within 7-14 days and from 14 to 28 days after allogeneic HSCT and refers to the step of engraftment of HSCs in the bone marrow niches to reconstitute immunity. Under optimal circumstances, the recipient's immune system tolerates donor cell engraftment without non-engraftment or late graft failure. Further, improvements in engraftment kinetics reduce transplantation costs. “Treating” or “treatment of a disease or condition” refers to any act intended to ameliorate the health status of patients. “Treatment” can include, but is not limited to, alleviation or amelioration of one or more symptoms or conditions, diminishment of extent of disease, stabilization of the state of disease (e.g. maintaining a patient in remission), prevention of the disease or prevention of the spread of disease, delay or slowing of disease progression, amelioration or palliation of the disease state, diminishment of the reoccurrence of disease, and remission (whether partial or total). A treatment may include curative, alleviation or prophylactic effects. The term “prophylactic” may be considered as reducing the severity or the onset of a particular condition. “Prophylactic” also includes preventing reoccurrence of a particular condition in a patient previously diagnosed with the condition. “Therapeutic” may also reduce or delay the severity of an existing condition. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In one embodiment, treating a cancer includes inhibiting the growth or proliferation of cancer cells or killing cancer cells. In a particular embodiment, treating a cancer includes reducing the risk or development of metastasis. In another particular embodiment, treating a cancer may refer to the prevention of a relapse. Treating a cancer may also refer to maintaining a subject in remission.

As used herein, the terms “disorder” or “disease” refer to the incorrectly functioning organ, part, structure, or system of the body resulting from the effect of genetic or developmental errors, infection, poisons, nutritional deficiency or imbalance, toxicity, or unfavourable environmental factors. Preferably, these terms refer to a health disorder or disease e.g. an illness that disrupts normal physical or mental functions. More preferably, the term disorder refers to immune and/or inflammatory diseases that affect animals and/or humans. Preferably, the term disorder or disease refers to cancers, infectious diseases or immune diseases. The term “immune disease” or “auto-immune disease”, as used herein, refers to a condition in a subject characterized by cellular, tissue and/or organ injury caused by an immunologic reaction of the subject to its own cells, tissues and/or organs.

### Treatment

The term “SSRIs” or “Selective Serotonin Reuptake Inhibitors” are a class of drugs that are typically used as antidepressants in the treatment of major depressive disorder and anxiety disorders. The exact mechanism of action of SSRIs is unknown. SSRIs are believed to increase the extracellular level of the neurotransmitter serotonin by limiting its reabsorption (reuptake) into the presynaptic cell, increasing the level of serotonin in the synaptic cleft available to bind to the postsynaptic receptor. SSRIs is used herein in the plural form to designate each member of this drug family, accordingly, it will be easily understood that when related to the combinations according to the invention, SSRIs or Selective Serotonin Reuptake Inhibitors is used interchangeably with “at least one SSRI” or “at least one Selective Serotonin Reuptake Inhibitor”.

As used herein, the term “serotonin”, “5-hydroxytryptamine” or “5-HT” refers to a monoamine neurotransmitter which is highly conserved throughout evolution. The indoleamine molecule derives from the amino acid tryptophan. Serotonin is primarily synthesized by enterochromaffin cells found in in the gastrointestinal tract (GI tract). However, it is also produced in the central nervous system (CNS). Additionally, serotonin is stored in blood platelets and is released during agitation and vasoconstriction, where it then acts as an agonist to other platelets. 5-HT is involved in cognition, attention, emotion, pain, sleep, and arousal, to name a few examples. Yet only a small percentage of the body's 5-HT (˜5%) can be found in the mature brain of mammals: most (˜95%) is found synthesized in the gastrointestinal tract. The cellular effects of 5-HT are exerted through activation of any of 15 different receptors in 7 different classes (5-HT1 to 5-HT7). Through these receptors, 5-HT can produce opposing effects that add to the complexity of the serotonergic system.

For example, SSRIs are fluoxetine, citalopram, sertraline, paroxetine, escitalopram, fluvoxamine. By “fluoxetine”, it refers to (RS)-N-methyl-3-phenyl-3-propan-1-amine (CAS no. 54910-89-3) of formula (I) below:

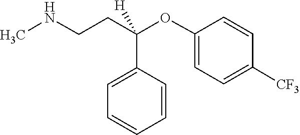 [SMILES: CNCC[C@@H](Oc1ccc(C(F)(F)F)cc1)c1ccccc1]

By “citalopram”, it refers to (RS)-1-[3-(dimethylamino)propyl]-1-(4-fluorophenyl)-1,3-dihydro[3,4]benzofuran-5-carbonitrile (CAS no. 59729-33-8) of formula (II) below:

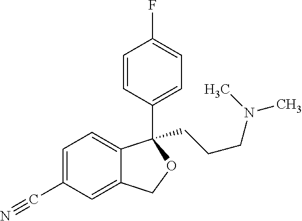 [SMILES: CN(C)CCC[C@]1(c2ccc(F)cc2)OCc2cc(C#N)ccc21]

By “Sertraline”, it refers to (1S,4S)-4-(3,4-dichlorophenyl)-N-methyl-1,2,3,4-tetrahydronaphtalen-1-amine (CAS no. 79617-96-2) of formula (Ill) below:

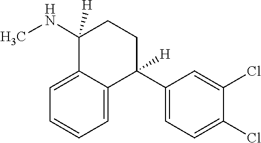 [SMILES: CN[C@H]1CC[C@@H](c2ccc(Cl)c(Cl)c2)c2ccccc21]

By “Paroxetine”, it refers to (3S,4R)-3-[(1,3-benzodioxol-5-yloxy)methyl]-4-(4-fluorophenyl)piperidine (CAS no. 61869-08-7) of formula (IV) below:

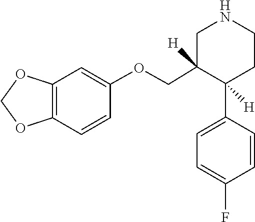 [SMILES: Fc1ccc([C@@H]2CCNC[C@H]2COc2ccc3c(c2)OCO3)cc1]

By “escitalopram”, it refers to (1S)-1-[3-(dimethylamino)propyl]-1-(4-fluorophenyl)-3H-2-benzofuran-5-carbonitrile (CAS no. 128196-01-0) of formula (V) below:

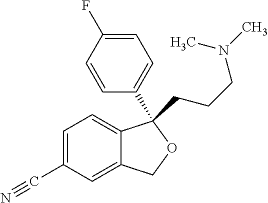 [SMILES: CN(C)CCC[C@@]1(c2ccc(F)cc2)OCc2cc(C#N)ccc21]

By “Fluvoxamine”, it refers to (E)-2-[(5-methoxy-1-[4-(trifluoromethyl)phenyl]pentyliden)amino]oxyethanamine (CAS no. 54739-18-3) of formula (VI) below:

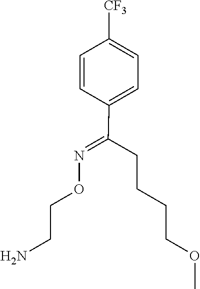 [SMILES: COCCCC/C(=N\OCCN)c1ccc(C(F)(F)F)cc1]

The term “hematopoietic growth factors” refers to factors who regulate the differentiation and the proliferation of particular progenitor cells. Granulocyte-macrophage colony-stimulating factor and granulocyte CSF are given to stimulate white blood cell formation in cancer patients who are receiving chemotherapy, which tends to kill their bone marrow cells as well as the cancer cells. Thrombopoietin shows great promise for preventing platelet depletion during chemotherapy. CSFs and thrombopoietin also improve the outcome of patients who receive bone marrow transplants. For example, hematopoietic growth factors are erythropoietin (EPO), thrombopoietin (TPO), granulocyte colony-stimulating factor (G-CSF), granulocyte-macrophage colony-stimulating factor (GM-CSF), macrophage colony-stimulating factor (M-CSF), platelet-derived growth factor (PDGF), interleukin 3 (IL-3), interleukin 6 (11-6). erythropoietin or EPO is a glycoprotein cytokine secreted by the kidney in response to cellular hypoxia; it stimulates red blood cell production (erythropoiesis) in the bone marrow. Thrombopoietin (TPO), also known as megakaryocyte growth and development factor (MGDF), refers to a protein that in humans is encoded by the TPO gene. Particularly, it is a glycoprotein hormone produced by the liver and kidney which regulates the production of platelets. It stimulates the production and differentiation of megakaryocytes, the bone marrow cells that bud off large numbers of platelets. Granulocyte-colony stimulating factor (G-CSF or GCSF), also known as colony-stimulating factor 3 (CSF 3), is a glycoprotein that stimulates the bone marrow to produce granulocytes and stem cells and release them into the bloodstream. Granulocyte-macrophage colony-stimulating factor (GM-CSF), also known as colony-stimulating factor 2 (CSF2), is a monomeric glycoprotein secreted by macrophages, T cells, mast cells, natural killer cells, endothelial cells and fibroblasts that functions as a cytokine. Unlike granulocyte colony-stimulating factor, which specifically promotes neutrophil proliferation and maturation, GM-CSF affects more cell types, especially macrophages and eosinophils. M-CSF (CSF 1) stimulates hematopoietic stem cells and progenitors to produce increased myeloid immune cells that have a role against diverse pathogens. Platelet-derived growth factor (PDGF) is one of numerous growth factors that regulates cell growth and division. In particular, PDGF plays a significant role in blood vessel formation. Interleukin 3 (IL-3) is a T cell-derived pluripotent hematopoietic colony-stimulating factor required for the survival and proliferation of primitive hematopoietic progenitor cells. Interleukin 6 (IL-6) is an interleukin that acts as both a pro-inflammatory cytokine and an anti-inflammatory myokine. “Hematopoietic growth factors” is used herein in the plural form to designate each member of this drug family; accordingly, it will be easily understood that when related to the combinations according to the invention, “hematopoietic growth factors” is used interchangeably with “at least one hematopoietic growth factor” or “at least one at least one hematopoietic growth factor”.

Despite the teachings of the prior art, according to which selective serotonin reuptake inhibitors (SSRIs) are typically used as antidepressants in the treatment of major depressive disorder, the inventors surprisingly found that the combined use of selective serotonin reuptake inhibitors (SSRIs) and hematopoietic growth factors generates totally unexpected effects and synergistic effects, thus making it possible to obtain better treatment of cytopenia related to hematopoietic diseases and especially after a chemotherapy or radiotherapy. In a particular aspect, said combination generates synergistic effects and reduce the duration of chemotherapy-induced aplasia.

Analysis of in vivo experiments after orally administration of fluoxetine in a human cohort of patients subjected to HSC transplants surprisingly show that the length of aplasia after myeloablative chemotherapy was considerably reduced by at least two days, which is of peculiar interest in regard with the common duration of around 10 to 13 days that is usually observed.

Accordingly, the present invention relates to the use of a combination of at least one selective serotonin reuptake inhibitor (SSRI) and at least one hematopoietic growth factor as drug. In a particular embodiment of the invention, said combination is particularly suited after chemotherapy treatment, especially to improve and reduce aplasia length, said chemotherapy being administered either to prepare graft with HSCs or in the course of cancer treatment. By “Aplasia”, it is referred to cytopenia occurring on all hematopoietic lineages. After chemotherapy, aplasia relates to a temporary defective production and/or maturation of cells of all hematopoietic lineages. It represents the time period during which there is a defective development of hematopoietic stem or progenitor cells, or more particularly the time period during which the level of at least one or several hematopoietic lineage(s) is under commonly accepted levels in scientific community (Common Terminology Criteria for Adverse Events (CTCAE) v5.0). Especially, after chemotherapy, there is a temporary blockage of the activity of bone marrow or hematopoiesis inducing a decrease of the production of blood cells. This period of time is particularly critical because the patients are more vulnerable, exhausted, in need of transfusion, which results in a reduced quality of life and also, when immune cells are low, a high risk of infection.

In another embodiment, the invention relates to a combination of at least one selective serotonin reuptake inhibitor and at least one hematopoietic growth factor for its use in the treatment of hematopoietic diseases, especially for patient in need of hematopoietic stem cell transplantation (HSCT).

Previous HSCT, a conditioning treatment is administered to patient in need of said transplantation to eliminate the underlying disease, create space for the new marrow and prevent rejection of the new bone marrow. Once the conditioning treatment has begun, patients usually need to be in protective isolation to help prevent infection. Protective isolation means that it is necessary for the patient to remain in the hospital room or ward most of the time. Protective isolation continues throughout transplant and for about three weeks post-transplant, until the patient's condition and white blood cell count have improved to a satisfactory level. There is a variety of conditioning regimens that involve chemotherapy alone, a total irradiation of the body (TBI), or a combination of chemotherapy and total body irradiation (TBI).

Chemotherapy: Patients receive chemotherapy drugs prior to the blood and marrow transplant. The chemotherapy is given in high doses in order to eliminate the disease or cancer. In the case of an allogeneic (donor), chemotherapy suppresses the immune system to allow the transplanted bone marrow to undergo a process called engraftment.

Total Body Irradiation (TBI): In some cases patients receive radiation therapy in addition to chemotherapy during their conditioning treatment. Like chemotherapy, total body irradiation (TBI) is used to eliminate the disease and in the case of donor or allogeneic transplant, to suppress the patient's immune system in preparation for the transplanted stem cells. In some instance also, TBI is used alone for conditioning patients.

Accordingly, in a particular embodiment, the invention relates to a combination of at least one serotonin reuptake inhibitor (SSRI) and at least one hematopoietic growth factor according to the invention, for use in improving hematopoietic stem and progenitor cell regeneration in a subject who has been subjected to high doses and/or myeloablative chemotherapy and/or TBI in order to eliminate disease or cancer and/or ensure stem cell engraftment.
In the context of the present invention, hematopoietic diseases comprise:
   hematopoietic diseases responsible for development of anemia of central or peripheral mechanism,
   hematopoietic diseases responsible for development of thrombopenia of central or peripheral mechanism,
   hematopoietic diseases responsible for development of neutropenia of central or peripheral mechanism.

In a particular embodiment, hematopoietic diseases responsible for development of anemia comprise malignant hemopathies, myelodysplastic syndromes, hemolytic anemia, hemoglobinopathies, or aplastic anemia.

In another particular embodiment hematopoietic diseases responsible for development of thrombopenia comprise malignant hemopathies, myelodysplastic syndromes, inherited or acquired peripheral thrombopenia, or aplastic anemia.

In another particular embodiment, hematopoietic disease is selected from the group consisting in malignant hemopathies, myelodysplastic syndromes, hemolytic anemia, hemoglobinopathies, aplastic anemia, inherited or acquired peripheral thrombopenia, inherited or acquired neutropenia, and aplastic anemia.

In another particular embodiment hematopoietic diseases responsible for development of neutropenia comprise malignant hemopathies, myelodysplastic syndromes, inherited or acquired neutropenia, or aplastic anemia.

In another particular embodiment, said combination is administered to a patient for the treatment of cytopenia. By “cytopenia”, it refers to context when one or more of patient blood cell types is lower than it should be. Blood consists of three main parts. Red blood cells, also called erythrocytes, carry oxygen and nutrients around the body. White blood cells, or leukocytes, fight infection and battle unhealthy bacteria. Platelets are essential for clotting. If any of these elements are below typical levels, patients have cytopenia. Several types of cytopenia exist:
Anemia occurs when your red blood cells are low.
Leukopenia is a low level of white blood cells.
Thrombocytopenia is a deficiency of platelets.
Pancytopenia is a deficiency of all three myeloid lineages in the blood.

But, the possible causes of cytopenia are complex and varied. Among these causes are peripheral destruction, infections, and side effects of medications or treatments. Two types of cytopenia that are related to the underlying cause of the low blood cell count are autoimmune cytopenia and refractory cytopenia. Autoimmune cytopenia is caused by an autoimmune disease. the body produces antibodies that fight against the healthy blood cells, destroying them and preventing you from having adequate blood cell counts. Refractory cytopenia occurs when the bone marrow does not produce mature, healthy blood cells. This may be the result of a group of cancers, such as leukemia or another bone marrow condition.

Consequently, in a particular embodiment, the invention relates to a combination of at least one selective serotonin reuptake inhibitor (SSRI) and at least one hematopoietic growth factor for use in treating a cytopenia related to a hematopoietic disease selected from the group consisting in malignant hemopathies, myelodysplastic syndromes, hemolytic anemia, hemoglobinopathies, aplastic anemia, inherited or acquired peripheral thrombopenia, inherited or acquired neutropenia, and aplastic anemia.

In another particular embodiment, the invention relates to a combination of at least one selective serotonin reuptake inhibitor (SSRI) and at least one hematopoietic growth factor for use according for use in treating a cytopenia related to a hematopoietic disease selected from the group consisting in malignant hemopathies, hemolytic anemia, hemoglobinopathies, inherited or acquired peripheral thrombopenia, inherited or acquired neutropenia, and aplastic anemia.

In a more particular embodiment, the invention relates to a combination of at least one selective serotonin reuptake inhibitor (SSRI) and at least one hematopoietic growth factor for use in treating a cytopenia related to a myelodysplastic syndrome.

In a particular embodiment of the invention, the combination of at least one selective serotonin reuptake inhibitor (SSRI) and at least one hematopoietic growth factor improves hematopoietic stem and progenitor cells regeneration. Hence, this combination is preferably a drug for its use in treating diseases or conditions involving or requiring hematopoietic stem and progenitor cells regeneration. Accordingly, said combination reduces the duration of aplasia and so, the risk of infection for patient having received a hematopoietic stem cell transplantation. Also, by reducing the duration of aplasia, subject quality of life is greatly improved as upon combinatory treatment of the invention said subject is not anymore in need for transfusion and does not experience exhaustion related to aplasia. The reduction of the duration of aplasia can be related to a direct or indirect effect of the combinations according to the invention on hematopoietic stem and progenitor cells, including improvement of their survival, proliferation and/or maturation. This can relate to the field of regenerative medicine, and, in hematology, is particularly interesting in the context of stem cell transplantation.

In another preferred embodiment, the combination of at least one selective serotonin reuptake inhibitor (SSRI) and at least one hematopoietic growth factor comprises at least one selective serotonin reuptake inhibitor (SSRI) selected from the group consisting in fluoxetine, citalopram, sertraline, paroxetine, escitalopram, fluvoxamine.

In another preferred embodiment, the combination of at least one selective serotonin reuptake inhibitor (SSRIs) and least one hematopoietic growth factor comprises at least one hematopoietic growth factor selected from the group consisting in erythropoietin (EPO), thrombopoietin (TPO), G-CSF, IL-3, 11-6, GM-CSF, M-CSF, PDGF.

In a particular embodiment, combinations of at least one selective serotonin reuptake inhibitor (SSRI) and at least one hematopoietic growth factor according to the present invention are particularly suited to improve hematopoietic stem and progenitor cells regeneration. Accordingly, inventors shown that said combination of at least one selective serotonin reuptake inhibitor (SSRI) and at least one hematopoietic growth factor reduces the duration of aplasia and also, improves Hematopoietic Stem Cells mobilization and/or engraftment function when administered to a subject suffering from aplasia or in need of improvement of Hematopoietic Stem Cells mobilization and/or engraftment function. Therefore, combinations of the invention are particularly of interest for patients with cytopenia inducing disorders. Preferably, said combinations are administered to patient suffering of cytopenia secondary to hematopoietic diseases and/or chemotherapy and/or radiotherapy.

In a preferred embodiment of the present invention, said combinations are used to treat hematopoietic disease in an allograft or autograft context. In particular, said autograft is a hematopoietic stem cells (HSCs)-autograft, more particularly with HSCs obtained from peripheral blood cells originating from the patient. In another object, said allograft is a hematopoietic stem cells (HSCs)-allograft, for which it can be made use of either peripheral blood, bone marrow or cord cells from a donor, i.e. from a subject who is not the receiver of the graft. Thus, in this embodiment, the combinations of the invention are administered in case of chemotherapy-induced and/or irradiation-induced (TBI) aplasia which is provoked before autologous or heterologous transplant, especially autologous hematopoietic stem cells (HSCs) transplant or heterologous hematopoietic stem cells (HSCs) transplant. In the autograft context, combinations of the invention are particularly advantageous when administered to the patient, who is also the donor of HSCs, for both improving hematopoietic stem and progenitor cells mobilization, and improving engraftment function in said patient. In the allograft context, said combinations are particularly advantageous when administered to the donor, to improve hematopoietic stem and progenitor cells mobilization and, in the recipient of the graft (i.e. the patient suffering from aplasia or cytopenia), for improving engraftment function in said recipient.

In an embodiment of the invention, said combinations according to the invention are of particular interest for cancer defined as hematopoietic malignancies. Accordingly, the present invention relates to a combination of at least one selective serotonin reuptake inhibitor (SSRI) and at least one hematopoietic growth factor for use in treating myelodysplastic syndrome, inherited or acquired neutropenia, or aplastic anemia. In a particular embodiment, the present invention relates to a combination of at least one selective serotonin reuptake inhibitor (SSRI) and at least one hematopoietic growth factor for use in treating a myelodysplastic syndrome selected in the group consisting in refractory anemia (RA), refractory anemia with ring sideroblasts (RARS), refractory anemia with excess blasts (RAEB), refractory anemia with excess blasts in transformation (RAEB-T), and chronic myelomonocytic leukemia (CMML).

In the context of the myelodysplastic syndrome, a combination according to the invention comprising at least one selective serotonin reuptake inhibitor (SSRI) and at least EPO is preferred. Alternately, in that specific context, another preferred combination according to the invention comprises at least one serotonin reuptake inhibitor (SSRI) and at least G-CSF. Alternately, in that specific context, another preferred combination comprises at least one serotonin reuptake inhibitor (SSRI) and at least TPO.

As shown in the experimental section, combination according the invention is also particularly suited to treat ineffective erythropoiesis, through the promotion of erythropoiesis and/or the correction of ineffective erythropoiesis. Accordingly, in a preferred embodiment, the invention relates to a combination at least one selective serotonin reuptake inhibitor (SSRI) and at least one hematopoietic growth factor for its use in treating anemia, such as a correction of anemia, in myelodysplastic syndromes. In the context of erythropoiesis disorders, a combination according to the invention comprising at least one selective serotonin reuptake inhibitor (SSRI) and erythropoietin (EPO) is particularly preferred.

Also, combinations of the invention are particularly suited to treat hemostasis disorders and/or thrombosis disorders. Accordingly, in an embodiment the invention relates to a combination according to the invention comprising at least one selective serotonin reuptake inhibitor (SSRI) and at least one hematopoietic growth factor for use in treating hemostasis disorders and/or thrombosis disorders. In that context, in a preferred embodiment, the invention relates to a combination according to the invention comprising at least one selective serotonin reuptake inhibitor (SSRI) and at least one platelet-derived growth factor (PDGF) for use in treating hemostasis disorders and/or thrombosis disorders.

Combinations according to the invention are also particularly suited to treat auto-immune disorders and/or inflammation disorders that induce and/or are associated with aplasia or cytopenia. Accordingly, the invention relates to a combination according to the invention comprising at least one selective serotonin reuptake inhibitor (SSRI) and at least one hematopoietic growth factor for use in treating said aplasia or cytopenia in the context of auto-immune disorders and/or inflammation disorders. In that context, in a preferred embodiment, the invention relates to a combination at least one serotonin reuptake inhibitor (SSRI) and at least the granulocyte-colony stimulating factor (G-CSF) for use in treating said aplasia or cytopenia.

The invention also relates to methods of improving hematopoietic stem and progenitor cell regeneration in a subject in need thereof comprising the administration to said subject at least one selective serotonin reuptake inhibitor (SSRI) and at least one hematopoietic growth factor as described above. Said method, as exposed above, is of particular interest for treating subjects suffering from cytopenia, which constitutes a particular embodiment of said method. Even more particularly said subject to be treated in said method is suffering from cytopenia secondary to chemotherapy or radiotherapy. In another particular embodiment said subject is suffering from cytopenia secondary to a hematopoietic disease, which are described above.

Another object of invention the resides in a method of improving hematopoietic stem and progenitor cell regeneration in a subject in need for hematopoietic stem cell transplantation as described above. Indeed, combinations according the invention are useful improving engraftment, function, and/or mobilization of HSCs and results in the shortening of aplasia subsequent to chemotherapy and/or TBI treatment applied to prepare the subject to the graft. In particular said method is a conditioning method of a subject to be subjected to HSCs graft comprising the steps of:
i) Administering to said patient at least one SSRI,
ii) Administering to said patient a chemotherapy and/or TBI to eliminate the disease and in the case of donor or allogenic transplant, to suppress the patient's immune system in preparation for the transplanted stem cells,
iii) Administering to the subject at least one hematopoietic growth factor.

In this method of conditioning a subject to be subjected to HSCs graft, said at least one selective serotonin reuptake inhibitor (SSRI) and at least one hematopoietic growth factor are those as described previously.

In a particular embodiment, as exposed above the step i) begins and lasts several days before the step ii), for example in order for the SSRI to reach the proper plasmatic upon aplasia establishment and/or administration of hematopoietic growth factor according to step iii). Preferably step i) is performed from 2 days, 3 days, 4 days, 5 days, 6 days, more preferably 7 days before performing step ii) of administering a chemotherapy and/or TBI to eliminate the disease and in the case of donor or allogenic transplant, to suppress the patient's immune system in preparation for the transplanted stem cells. In another particular embodiment step iii) of administering a hematopoietic growth factor starts from day 2 following step ii) more preferably from day 3, day 4, day 5, day 6, or even preferably from day 7 following step ii) and lasts, together with the administration of SSRI of step i) till an improvement of count of at least one type of blood cells, more preferably till the end of aplasia induced in the subject. In an even more particular embodiment, depending of the hematopoietic growth factor formulation (e.g. pegfilgrastim, a PEGylated form of G-CSF), said hematopoietic growth factor is administered once from day 3, day 4, day 5, day 6, or even preferably from day 7 following step ii). The skilled in the art will know to determine the most appropriate administration.

### Administration

In the course on combinatory treatment according to the invention, the administration of the at least one selective serotonin reuptake inhibitor (SSRI) and of the at least hematopoietic growth factor can be simultaneous or sequential, these terms being defined above.

Whether the administration is simultaneous or sequential, said at least one selective serotonin reuptake inhibitor (SSRI) and at least one hematopoietic growth factor, can be administered by any appropriate route of administration, oral route being preferred when possible. In a preferred embodiment, the at least one selective serotonin reuptake inhibitor (SSRI) and at least one hematopoietic growth factor, are both administered.

Although there is to date no combined formulation of a selective serotonin reuptake inhibitor (SSRI) and a hematopoietic growth factor, the development of such a formulation would make it possible to simplify the simultaneous administration of these two active ingredients for patients.

The synergistic effects shown by the inventors make it possible to reduce significantly the duration of aplasia. In the prior art, the doses of fluoxetine administered to patients suffering from mood disorders and especially depressive disorders are 20 mg/kg/day orally. Furthermore, the dose and administration mode are known for every SSRIs currently prescribed, such as fluoxetine, citalopram, sertraline, paroxetine, escitalopram, fluvoxamine. In an embodiment, the doses of SSRIs used in the combinations and therapies according to the invention are identical or equivalent to those usually prescribed in their original medical indication.

Also, hematopoietic growth factors are well known in the art and are frequently used in cytopenia resulting from various hematopoietic and non-hematopoietic disorders or chemotherapy. In an embodiment identical or equivalent doses and dosage as for their original indication of hematopoietic growth factors are use in combinations and therapies according to the invention. For example, and without limiting the scope of the present invention, EPO is used in anemia of hematopoietic origin as follows: darbopoetine a is administered in 150 to 300 μg/week or 500 μg/3 weeks, epoietine β is administered in 30 000 UI/week; epoietine a is administered in 40 000 UI/week. For example, TPO agonists are used in thrombopenia of hematopoietic origin as follows: eltrombopag is administered in 25 to 75 mg/day; romiplostim is administered in 250 to 500 μg/week and maximum 10 μg/kg/week.

For example, G-CSF are used of in neutropenia hematopoietic origin as follows: lenograstim is administered in 13 or 34 MUI/ml, 1 injection/day; filgrastim is administered in 30 or 48 MUI/ml, 1 injection/day; tevagrastim is administered in 30 or 48 MUI/ml, 1 injection/day; pegfilgrastim is administered in 6 mg once.

As mentioned above, the at least one SSRI and the at least one hematopoietic growth factor of the combination according to the invention may be administered concomitantly, either in the same or a different pharmaceutical formulation or sequentially. If there is separated and/or sequential administration, administration scheme should be designed to obtain the most effective combination. For example, the delay in administering the second (or additional) active ingredient should not be such as to lose the benefit of the efficacious effect of the combination of the active ingredients. The skilled in the art knows the pharmacodynamics for these drugs to be used in the combinations of the invention as these drugs are currently prescribed drugs in other medical indications. Consequently, the dose and posology for each drug of the combination according to the invention can be adapted to PK/PD features of said drugs and to the intended use of the combinatory treatment according to the invention, in order that each drug, within the combinatory treatments of the invention, be fully effective.

For example, when related to treating aplasia resulting from the conditioning treatment of a HSCs graft receiver and to favouring the engraftment, a preferred administration scheme is a separated administration of the at least one SSRI and the at least one hematopoietic growth factor, so that each drug reaches its maximal effective plasmatic concentration at the time of the conditioning treatment and/or onset of aplasia. Accordingly, in this specific embodiment, the at least one SSRI is preferably administered on a regular basis before myeloablative conditioning treatment, even more particularly 3 days, 4 days, 5 days, 6 days, more preferably 7 days before the conditioning treatment in order that said SSRI reaches its maximal effective plasmatic concentration at the right time, and then hematopoietic growth factor is then added to the treatment scheme. In a particular embodiment, hematopoietic growth factor is administered 1, 2, 3 or 4 days after the conditioning treatment. In a more particular embodiment, for autologous stem cell transplant G-CSF or Peg GCSF is started at day +5 after the stem cell transplantation. In an even more particular embodiment, in specific chemotherapy protocols, G-CSF is started between 2-3 days after the end of the chemotherapy and until absolute number of neutrophils is >1000/mm3.This specific mode of administration is exemplified in the experimental section and results in the significant shortening of aplasia duration resulting from a more successful engraftment and/or mobilization of HSCs.

### Pharmaceutical Composition

In a second aspect, the invention also relates to a pharmaceutical kit comprising selective serotonin reuptake inhibitors (SSRIs) and hematopoietic growth factors.

In the pharmaceutical kit according to the invention, selective serotonin reuptake inhibitors (SSRIs) and hematopoietic growth factors can be present in the kit in a single formulation associating the two active ingredients 1) selective serotonin reuptake inhibitors (SSRIs) and 2) hematopoietic growth factors, or of two distinct formulations each comprising one of the active ingredients (thus allowing simultaneous or sequential administration).

Further aspects and advantages of the invention will be disclosed in the following examples, which should be considered illustrative.

### EXAMPLE

### Materials and Methods.

Animal Procedures

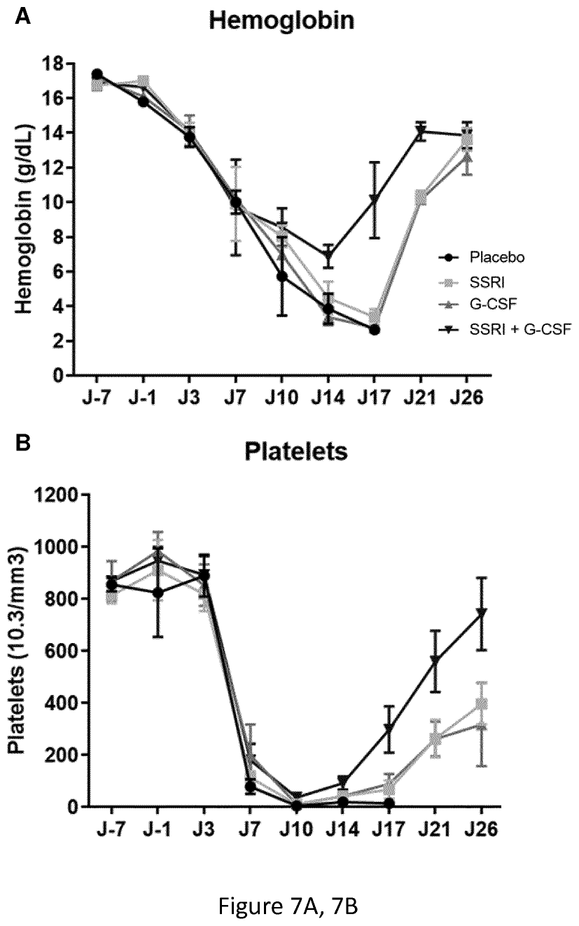

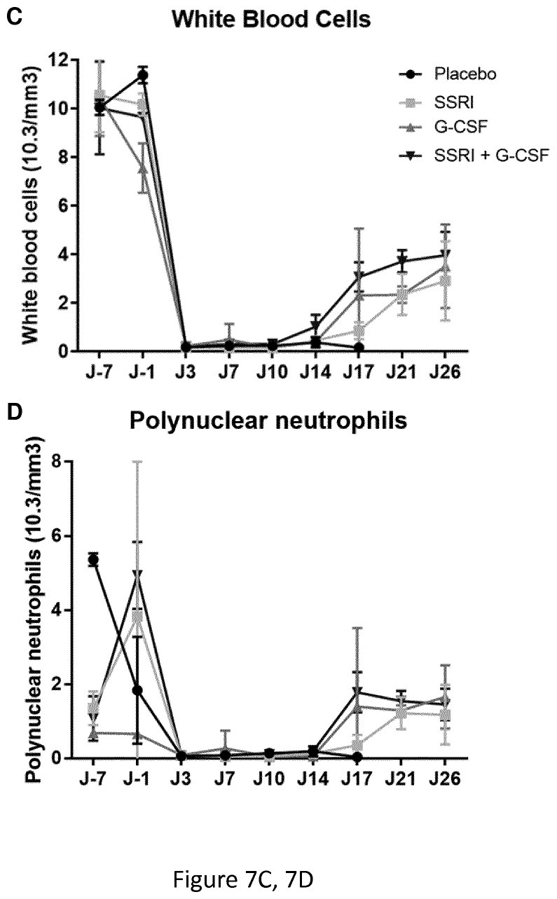

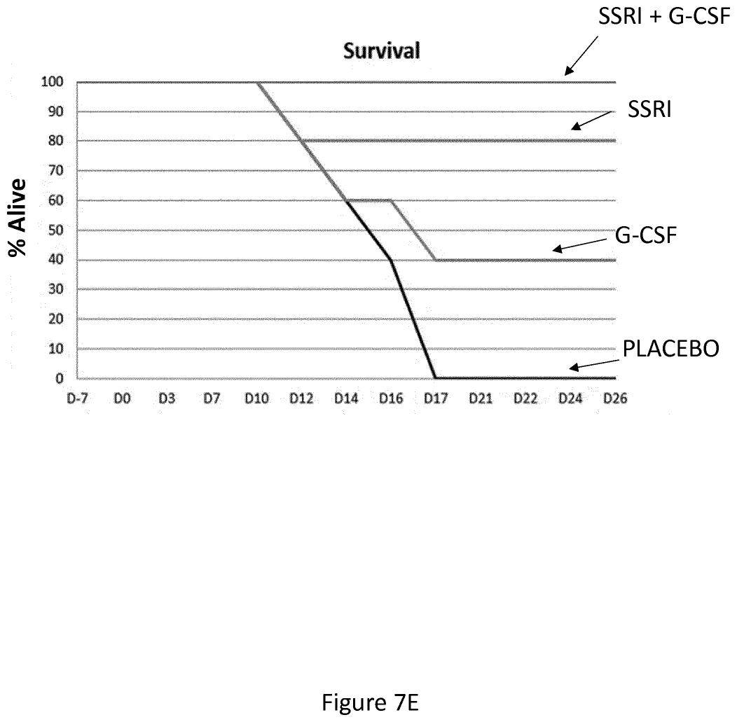

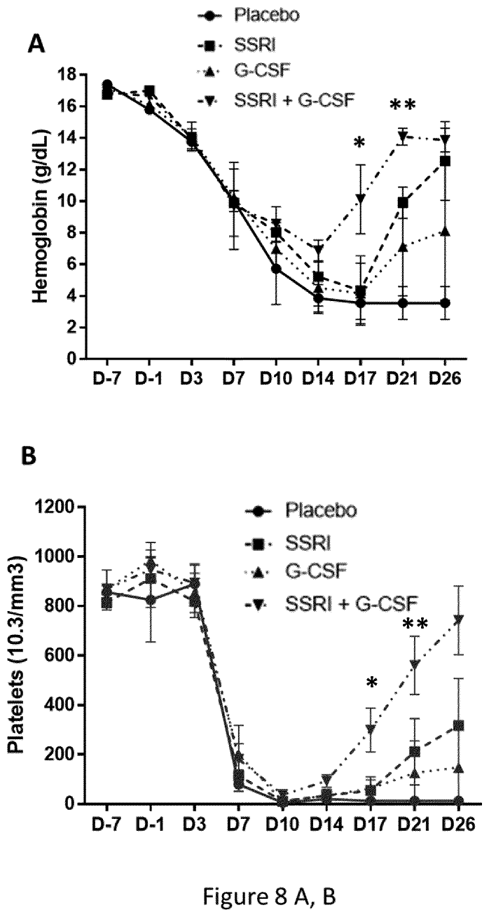

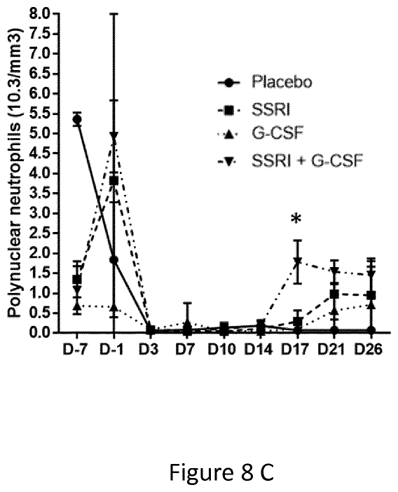

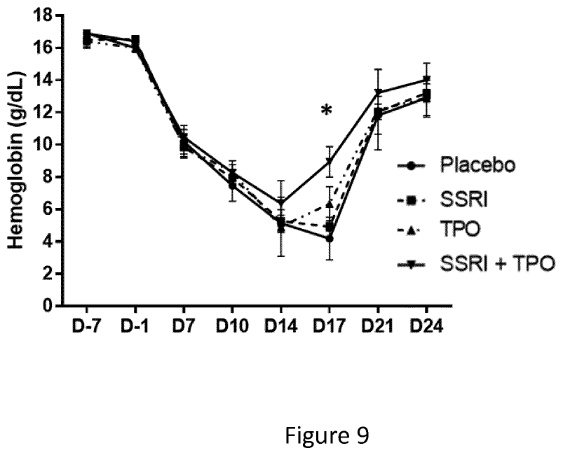

Tph1^{-/-} mice were generated as described (Côté et al., 2003). Tph1^{-/-} and WT animals were derived from pure C57BL/6J genetic backgrounds. For some experiments, C57/bl6 mice were purchased from Janvier Labs. Tph1^{+/-} mice were also used to mimic a physiological situation and relate findings disclosed herein to human health issues, as individuals with lower 5-HT level may be more at risk to develop myelodysplastic syndromes related anemia.
Two experiments of bone marrow transplantation were performed: one comprising 5 animals per group, (results presented on FIG. 7) the other performed on between 10 to 13 animals per group (FIG. 8). Same experiment was also performed with fluoxetine as SSRI and with TPO as hematopoietic growth factor. Same results were obtained as illustrated in FIG. 9.

Animal experiments were performed according to the recommendations of the French Institutional Committee.

Blood Counts

To perform complete blood counts, peripheral blood from the tail vein was collected in EDTA tubes and analyzed using an electronic hematology particle counter (IDEXX Procyte).

Fluoxetine Treatment

Fluoxetine was administrated orally (40 mM in water bottle (Prozac® 20 mg/5 mL solution) to WT mice (males 8-10 weeks old C57/bl6 purchased form Janvier labs) for 7 days before inducing anemia.

Hematopoietic Growth Factor Treatment

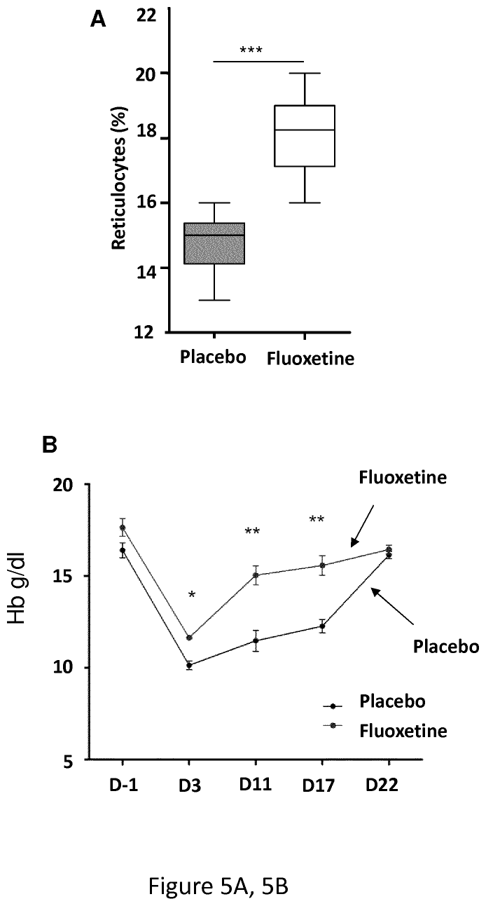

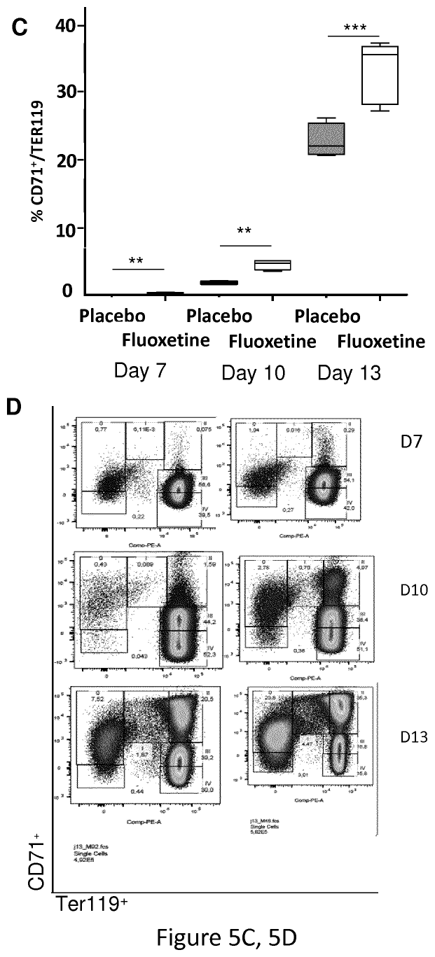

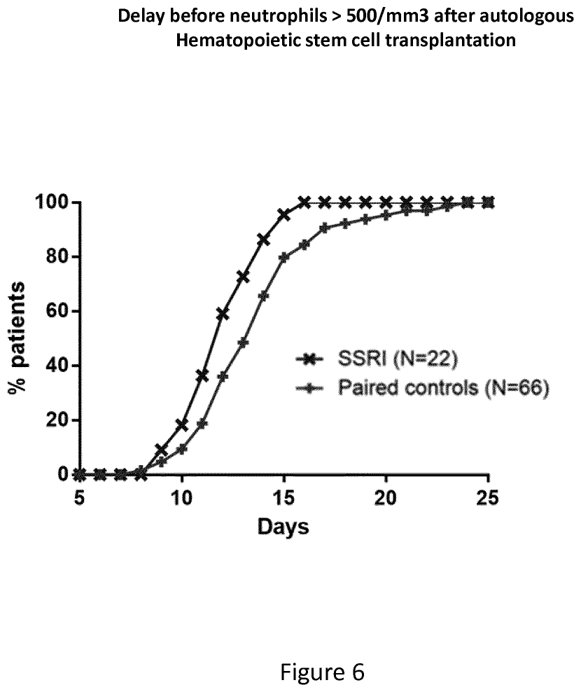

G-CSF treatment was administered subcutaneously daily from day 4 after irradiation and until resolution of aplasia (day 17 in FIG. 5), at the dose of 200 μg/kg/day.

In bone marrow replacement experiments, TPO treatment was also tested alone or in combinations according to the invention. TPO treatment was administered subcutaneously daily from after the day of irradiation and until resolution of aplasia (day 24 in FIG. 9), at the dose of 8 μg/kg/day.

Sub-lethal Irradiation

Sub-lethal irradiation was induced by submitting WT mice to 1.09 gy during 4 minutes (WT C57/bl6 Males 8-10 weeks old purchased form Janvier labs). Sub-lethal irradiation was induced by submitting Tph1^{+/-} mice to 1.09 gy during 4 minutes (Males 8-10 weeks old).

Bone Marrow Transfer Experiment

Bone marrow transplantation was performed as described in D′Aveni et al., 2015.

Cell Culture

Human Cord Blood Erythroid in Vitro Cell Culture.

Erythroid cells were generated from CD34⁺ cord blood progenitor cells in serum-free medium in the presence of EPO (2 mU/ml)+IL-3 (10 ng/ml)+stem cell factor (SCF; 50 ng/ml) as previously described in Zermati et al., (2000) Exp. Hematol. 28:885-894.

Immunophenotyping of Murine Erythroid Precursors.

Cells from fetal livers or BM cells flushed from femur and tibia were resuspended in Hank's buffered saline before being passed through a 100-μM strainer. Cells were washed, counted, and immunostained at room T° in PBS with PE-conjugated anti-TER119, FITC-conjugated anti-CD71 and APC-conjugated anti-CD117 (c-Kit) (BD Biosciences) antibodies for 20 min and analyzed on a FACS Canto II coupled with Flowio software version X.0.7 (Tree Star, Ashland, Oreg.).

RT qPCR

Total RNA was extracted from bone marrow cells using the RNeasy Kit (Qiagen). Reverse transcription was performed using iScript™ Reverse Transcription Supermix for RT-qPCR (BioRad). Real time PCR was performed on a STEPONE™ cycling machine (Applied Biosystems) using oligos from Taqman (Life technologies). Two biological replicates were used for each condition. Data were analyzed by StepOne Plus RT PCR software v2.1 and Microsoft excel. β-actin, GAPDH and 18S transcript levels were used for normalisation of each target (=ΔCT). Real-time PCR CT values were analysed using the 2-(ΔΔCt) method to calculate the fold expression (ΔΔCT method).

Patients

In order to evaluate the impact of treatment with SSRI on chemotherapy-induced aplasia, data of all patients benefiting from autologous hematopoietic stem cell transplantation (HSCT) from 2010 to 2017 in the hematology department of Necker Hospital, Paris, France were screened. Data from 22 patients who were under therapy with SSRI for usual indications at the time of autologous HSCT retrospectively reviewed, and compared with 66 control patients (3 controls per patient) matched for confounding factors (age, sex, disease, response of disease before therapy, conditioning regimen, date of transplant and number of CD34+ cells injected). It is observed a reduced duration of aplasia (a criterion of which, commonly described in the art, being a number of polynuclear neutrophils below 500/mm³) in patients who were treated with SSRI at the time of autologous HSCT.

Statistical Analysis
Statistical analysis was performed using GraphPad Prism software. Throughout data are mean±SEM. Unpaired t-test and Pearson linear and non-linear regressions were used when appropriate. *P<0.05, **P<0.005, ***P<0.0005.

### Results

With regard to the role played by serotonin in red blood cells production, the pharmacological and genetic evidences presented demonstrate that a functional autocrine serotonergic network exists in both murine and human erythroid progenitors. Tph1, the rate-limiting enzyme for peripheral serotonin synthesis is a novel erythroid gene. Serotonin is regulating hematopoietic stem cell fate along the erythroid pathway. The cytokine erythropoietin (EPO) induces TPH1 expression and serotonin synthesis necessary for erythroid progenitor's survival and proliferation. Together, the data demonstrate the existence of a synergy between serotonin and EPO to stimulate human pro-erythroblast proliferation. In vivo findings presented herein imply that increasing the concentration of bone marrow serotonin available to erythroid progenitors could be a valuable therapeutic intervention for myelodysplastic anemia before leukemic onset. Thus, the use of selective serotonin reuptake inhibitors (SSRIs) such as fluoxetine, a common antidepressant may have important clinical benefit in anemia treatment in combination with EPO, especially in myelodysplastic syndromes.

With regard to the role played by serotonin in the bone marrow, it could be used as a mitogen to regulate hematopoietic stem/progenitor cells maintenance and regeneration after various types of injury. Inventors show that SSRI treatment could have an impact on the engraftment, function, and/or mobilization of HSCs. Data demonstrate a reduction in the duration of aplasia in patients treated with SSRI at the time of autologous HSCT. Similar to the data obtained on erythroid progenitors, inventors show that SSRIs in combination with growth factors (TPO, G-CSF, PDGF, 11-3, IL-6, M-CSF) can be used for treating patients presenting cytopenia due to hematopoietic diseases and patients in need of chemotherapy and more particularly to reduce length of chemotherapy-induced aplasia.

As shown on FIGS. 7, 8 and 9 the second round of experiments of bone marrow transplantation further illustrates the effectiveness of combinations according to the invention in favoring the reduction of length of aplasia resulting from conditioning treatment. Results are summarized in table 1 below

TABLE 1
At Day 17 from sublethal irradiation, % of mice with

| | Normal | | Normal | Normal |
|---|---|---|---|---|
| | Hemoglobin | Hemoglobin | neutrophil | platelet |
| Treatment | level^{¥} | level ≤8 g/dl | number^{†} | level^{¥} |
| Placebo | 0 | 100 | 0 | 0 |
| SSRI monotherapy | 0 | 100 | 20 | 0 |
| Hematopoietic growth | 0 | 100 | 10 | 0 |
| factor monotherapy | | | | |
| SSRI and | 30-20 | 0 | 100 | 50 |
| Hematopoietic growth | | | | |
| factor | | | | |

^{¥}Santos et al. (2016);
^{†}Raabe et al. (2011)

### Conclusion

To conclude, results presented show that the combination of selective serotonin reuptake inhibitors (SSRIs) and hematopoietic growth factors can be used as drug and particularly for treating hematopoietic diseases and/or cytopenia. The present invention in particularly suited for patients in need of chemotherapy and more particularly to reduce length of chemotherapy-induced aplasia.

### REFERENCES

L. B. et al. Comparison of haematological recovery times and supportive care requirements of autologous recovery phase peripheral blood stem cell transplants, autologous bone marrow transplants and allogeneic bone marrow transplants. Bone marrow transplantation 9, 277-284 (1992).

L. B., Dyson, P. G. & Juttner, C. A. Cell-dose effect in circulating stem-cell autografting. Lancet 2, 404-405 (1986).

J. P. Feighner, Mechanism of action of antidepressant medications, The Journal of clinical psychiatry 60 Suppl 461 4 (1999) 4-11; discussion 12-3.

V. Erspamer, Experimental research on the biological significance of enterochromaffin cells, Arch. Fisiol. 37 (1937) 156-159;

V. Erspamer, B. Asero, Identification of enteramine, the specific hormone of the enterochromaffin cell system, as 5-hydroxytryptamine, Nature 169 (4306) (1952) 800-801

S. N. Spohn, G. M. Mawe, Non-conventional features of peripheral serotonin signalling—the gut and beyond, Nature reviews, Gastroenterol. Hepatol. 14 (7) (2017) 412-420

P. Amireault, D. Sibon, F. Cote, Life without peripheral serotonin: insights from tryptophan hydroxylase 1 knockout mice reveal the existence of paracrine/autocrine serotonergic networks, ACS Chem. Neurosci. 4 (1) (2013) 64-71.

Mosienko V, Beis D, Pasqualetti M, Waider J, Matthes S, Qadri F, Bader M, Alenina N.Behav,. Life without brain serotonin: reevaluation of serotonin function with mice deficient in brain serotonin synthesis. Brain Res. 2015 Jan. 15; 277:78-88.

Fouquet G, Coman T, Hermine O, Côté F. Serotonin, hematopoiesis and stem cells. Pharmacol Res. 2018 Aug. 11. pii: 51043-6618(18)30482-1.

Raabe, B. M., Artwohl, J. E., Purcell, J. E., Lovaglio, J., & Fortman, J. D. Effects of weekly blood collection in C57BL/6 mice. Journal of the American Association for Laboratory Animal Science: JAALAS, (2011). 50(5), 680-685.

SANTOS EW; Cunha de OLIVEIRA D; HASTREITER A; da SILVA GB; Soares de Oliveira BELTRAN J; TSUJITA M; CRISMA AR; NEVES P; FOCK RA; BORELLI P Hematological and biochemical reference values for C57BL/6, Swiss Webster and BALB/c mice. Braz. J. Vet. Res. Anim. Sci. (2016) v. 53, n. 2, p. 138-145

1. A combination of at least one selective serotonin reuptake inhibitor (SSRI) and at least one hematopoietic growth factor effective to improve hematopoietic stem and progenitor cell regeneration in a subject in need thereof.

2. The combination of at least one selective serotonin reuptake inhibitor (SSRI) and at least one hematopoietic growth factor according to claim 1, wherein said subject is suffering from cytopenia secondary to chemotherapy or radiotherapy.

3. The combination of at least one selective serotonin reuptake inhibitor (SSRI) and at least one hematopoietic growth factor according to claim 1, wherein said subject is suffering from cytopenia secondary to a hematopoietic disease.

4. The combination of at least one serotonin reuptake inhibitor (SSRI) and at least one hematopoietic growth factors according to claim 1, wherein said subject is in need for hematopoietic stem cell transplantation.

5. The combination of at least one serotonin reuptake inhibitor (SSRI) and at least one hematopoietic growth factor according to claim 4, wherein said subject has been subjected to high doses and/or myeloablative chemotherapy and/or TBI in order to eliminate disease or cancer and/or ensure stem cell engraftment.

6. The combination of at least one selective serotonin reuptake inhibitor (SSRI) and at least one hematopoietic growth factor according to claim 3, wherein said hematopoietic disease is selected from the group consisting of malignant hemopathies, including myelodysplastic syndromes (MDS), aplastic anemia, myeloproliferative neoplasm, acute leukemia, and non-malignant hemopathies including, hemolytic anemia, hemoglobinopathies, inherited or acquired peripheral thrombopenia, inherited or acquired neutropenia.

7. The combination of selective serotonin reuptake inhibitor (SSRI) and at least one hematopoietic growth factor according to claim 6, wherein said hematopoietic disease is a myelodysplastic syndrome.

8. The combination of selective serotonin reuptake inhibitor (SSRI) and at least one hematopoietic growth factor according to claim 4, adapted for use in allograft or autograft context.

9. The combination of at least one selective serotonin reuptake inhibitor (SSRI) and at least one hematopoietic growth factor according to claim 8, wherein autograft context is a peripheral blood hematopoietic stem cells (HSCs)-autograft, or allograft context is a hematopoietic stem cells (HSCs)-allograft, said cells originating from bone marrow, cord blood or peripheral blood from a donor.

10. A combination of at least one selective serotonin reuptake inhibitor (SSRI) and at least one hematopoietic growth factor effective to improve hematopoietic stem and progenitor cells mobilization in a (donor or recipient), and/or engraftment function in (the recipient).

11. The combination of at least one selective serotonin reuptake inhibitor (SSRI) and at least one hematopoietic growth factor according to claim, 10 wherein hematopoietic stem cells are collected from the peripheral blood of said donor or recipient after mobilization.

12. The combination of at least one selective serotonin reuptake inhibitor (SSRI) and at least one hematopoietic growth factor for use according to claim 1, wherein said at least one selective serotonin reuptake inhibitor (SSRI) is selected from the group consisting of fluoxetine, citalopram, sertraline, paroxetine, escitalopram, fluvoxamine.

13. The combination of at least one selective serotonin reuptake inhibitor (SSRI) and at least one hematopoietic growth factor according to claim 1, wherein said at least one hematopoietic growth factor is selected from the group consisting of: erythropoietin (EPO), thrombopoietin (TPO), G-CSF, IL-3, Il-6, GM-CSF, G-CSF, PDGF, M-CSF.

14. The combination of at least one selective serotonin reuptake inhibitor (SSRI) and at least one hematopoietic growth factor according to claim 2, being further effective to reduce length of aplasia, transfusion needs, aplasia related infections, and improve quality of life of said subject.

15. A pharmaceutical kit comprising at least one selective serotonin reuptake inhibitor (SSRI) and at least one hematopoietic growth factor effective to improve hematopoietic stem and progenitor cell regeneration, in a subject in need thereof.

16. A method of treatment comprising administering to a subject in need thereof an effective amount of a pharmaceutical composition comprising at least one selective serotonin reuptake inhibitor (SSRI) and at least one hematopoietic growth factor to improve hematopoietic stem and progenitor cell regeneration in said subject.

17. The combination of at least one selective serotonin reuptake inhibitor (SSRI) and at least one hematopoietic growth factor according to claim 10, wherein said at least one selective serotonin reuptake inhibitor (SSRI) is selected from the group consisting of: fluoxetine, citalopram, sertraline, paroxetine, escitalopram, fluvoxamine.

18. The combination of at least one selective serotonin reuptake inhibitor (SSRI) and at least one hematopoietic growth factor according to claim 10, wherein said at least one hematopoietic growth factor is selected from the group consisting of: erythropoietin (EPO), thrombopoietin (TPO), G-CSF, IL-3, Il-6, GM-CSF, G-CSF, PDGF, M-CSF.
